(19) United States
(12) Patent Application Publication
(10) Pub. No.: US 2024/0208933 A1
(43) Pub. Date: Jun. 27, 2024

(54) CRYSTALLINE INHIBITORS OF CYSTEINE PROTEASES AND METHODS OF USE THEREOF

(71) Applicant: Pardes Biosciences, Inc., Larkspur, CA (US)

(72) Inventors: Ruiping Wang, Beijing (CN); Meiting Shi, Beijing (CN); Arnold D. Lee, Rancho Santa Fe, CA (US); David J. Turnquist, Arlington, MA (US)

(21) Appl. No.: 18/589,700

(22) Filed: Feb. 28, 2024

Related U.S. Application Data
(63) Continuation of application No. PCT/US2022/075746, filed on Aug. 31, 2022.
(60) Provisional application No. 63/239,020, filed on Aug. 31, 2021.

(30) Foreign Application Priority Data
Aug. 31, 2021 (CN) 202111007881.6

(51) Int. Cl. C07D 401/12; A61K 31/454; A61K 45/06

(52) U.S. Cl. CPC C07D 401/12; A61K 31/454; A61K 45/06

(57) ABSTRACT

The disclosure is in part directed to crystalline forms of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide and pharmaceutical compositions thereof.

### CROSS-REFERENCE TO RELATED APPLICATIONS

This application is a continuation of International Patent Application No. PCT/US2022/075746, filed Aug. 31, 2022, which claims the benefit of, and priority to, U.S. Patent Application No. 63/239,020, filed Aug. 31, 2021; and Chinese Application No. 202111007881.6, filed Aug. 31, 2021; the contents of each of which are incorporated herein by reference.

### BACKGROUND

The Coronaviridae family of viruses are enveloped, single-stranded, positive-sense RNA viruses and include 141 species that are classified into four genera according to their phylogenetic relationships: α-, β-, γ-, and δ-coronavirus. Coronaviruses (CoVs) are zoonotic viruses that infect a variety of animals from whales to birds, bats, cats, and humans. Typically, CoV infection results in mild to moderate respiratory tract infections; however, some CoV species are extremely virulent and can result in widespread fatality. Severe acute respiratory syndrome coronavirus (SARS-CoV) is a human CoV that was responsible for the first pandemic of the 21st century, infecting over 8,000 people with a 10% mortality rate. Middle East respiratory syndrome coronavirus (MERS-CoV) was identified in November 2012 and had since infected over 1,600 people in 26 countries with 36% mortality rate. More recently, COVID-19 (SARS CoV2) coronaviruses have raised a global pandemic. Therefore, it is important to identify coronavirus drug targets that can be utilized for the development of broad-spectrum anti-coronaviral therapeutics to combat infections of existing and emerging coronaviruses.

All CoVs express a >800 kDa replicase polyprotein that contains either two or three cysteine proteases, the papain-like protease(s) (PLPpro, or PLP1 and PLP2) and the 3C-like protease (3CLpro, nsp5, or Mpro). These proteases process the CoV replicase polyprotein by cleaving it into 16 non-structural proteins, which are responsible for a variety of aspects of CoV replication. The CoV 3CLpro is responsible for processing 11 cleavage sites within the replicase polyprotein and is essential for CoV replication, making it a highly valuable target for therapeutic development. The overall active site architecture and substrate recognition pockets are structurally conserved across CoV 3CLpros, increasing its attractiveness as a target for the development of broad-spectrum anti-CoV therapeutics. Moreover, high sequence conservation in the vicinity of active site among CoV 3CLpros from different coronavirus subclasses make them an excellent target for the development of broad-spectrum therapeutics for coronavirus infections. Accordingly, the development of CoV 3CLpro inhibitors is a promising path for the treatment of respiratory tract infections and related diseases.

Numerous studies on targeting the immediate zoonotic reservoirs of coronaviruses with small molecule inhibitors have helped inform structure-based design strategies aimed at creating molecular scaffolds that may aid in the development of therapeutics against coronaviral infection; however, there is currently no commercially available broad spectrum small molecule antiviral agents. Therefore, there is a critical need for the development of broad-spectrum CoV therapeutics to overcome limitations of traditional anti-CoV therapeutic development, as broad-spectrum therapeutics can be rapidly implemented upon zoonotic disease outbreak.

### SUMMARY

The present disclosure is directed, at least in part, to crystalline forms of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide and crystalline anhydrates, hydrates and solvates thereof.

For example, disclosed herein is a crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate, characterized by a powder X-ray diffraction pattern having a characteristic peak in degrees 2θ at about 5.4, for example, characterized by a powder X-ray diffraction pattern having characteristic peaks in degrees 2θ at about 3.3, 3.7, and 5.4, for example, characterized by a powder X-ray diffraction pattern having characteristic peaks in degrees 2θ at about 3.3, 3.7, 5.4, 7.0 and 7.2.

7-Chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide is, for example, a protease inhibitor, and is represented by:

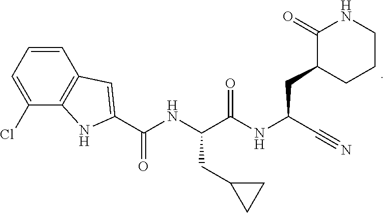 [SMILES: N#C[C@H](C[C@@H]1CCCNC1=O)NC(=O)[C@H](CC1CC1)NC(=O)c1cc2cccc(Cl)c2[nH]1]

Further contemplated herein is a pharmaceutical composition comprising a disclosed crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide and a pharmaceutically acceptable excipient, for example, a composition that is formulated for oral administration.

Also provided herein is a method of treating a viral infection in a patient in need thereof, comprising administering to the patient an effective amount of a disclosed crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide. For example, provided herein is a method of treating a viral infection in a patient in need thereof, comprising administering to the patient an effective amount of a pharmaceutical composition comprising a crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate.

### BRIEF DESCRIPTION OF THE DRAWINGS

FIG. 1 depicts the X-ray powder diffraction (XRPD) pattern of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methanol solvate (Form A).

FIG. 2 depicts the thermogravimetric analysis (TGA) profile of Form A (top) and the characterization of Form A by differential scanning calorimetry (DSC) (bottom).

FIG. 3 depicts the X-ray powder diffraction (XRPD) pattern of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate (Form B).

FIG. 4. depicts the thermogravimetric analysis (TGA) profile of Form B (top) and the characterization of Form B by differential scanning calorimetry (DSC) (bottom).

FIG. 5 depicts the X-ray powder diffraction (XRPD) pattern of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate (Form C).

FIG. 6 depicts the thermogravimetric analysis (TGA) profile of Form C (top) and the characterization of Form C by differential scanning calorimetry (DSC) (bottom).

FIG. 7 depicts the X-ray powder diffraction (XRPD) pattern of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl ethyl ketone solvate (Form D).

FIG. 8 depicts the thermogravimetric analysis (TGA) profile of Form D (top) and the characterization of Form D by differential scanning calorimetry (DSC) (bottom).

FIG. 9 depicts the X-ray powder diffraction (XRPD) pattern of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl isobutyl ketone solvate (Form E).

FIG. 10 depicts the thermogravimetric analysis (TGA) profile of Form E (top) and the characterization of Form E by differential scanning calorimetry (DSC) (bottom).

FIG. 11 depicts the X-ray powder diffraction (XRPD) pattern of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate (Form F).

FIG. 12 depicts the thermogravimetric analysis (TGA) profile of Form F (top) and the characterization of Form F by differential scanning calorimetry (DSC) (bottom).

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “crystalline form” refers to a crystal form or modification that can be characterized by analytical methods such as, e.g., X-ray powder diffraction (XRPD) and/or Differential scanning calorimetry (DSC). The crystalline compounds disclosed herein can exist in solvated as well as unsolvated forms with solvents such as water, ethanol, and the like. Unless otherwise indicated or inferred, it is intended that disclosed crystalline compounds include both solvated and unsolvated forms.

The term “treating” includes any effect, e.g., lessening, reducing, modulating, or eliminating, that results in the improvement of the condition, disease, disorder and the like.

The term “disorder” refers to and is used interchangeably with, the terms “disease,” “condition,” or “illness,” unless otherwise indicated.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system or animal, (e.g. mammal or human) that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “and/or” is used in this disclosure to mean either “and” or “or” unless indicated otherwise.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. The term “about” in the context of peaks at degrees 2θ means that there is an uncertainty in the measurements of the 20 of +0.5 (expressed in 2θ) or that there is an uncertainty in the measurements of the 20 of +0.2 (expressed in 2θ).

### I. Crystalline Forms

The present disclosure is directed, at least in part, to crystalline forms of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide.

### Form A

In certain embodiments, disclosed herein is a crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methanol solvate (referred to herein as “Form A”), characterized by a powder X-ray diffraction pattern that has a characteristic peak in degrees 2θ at about 9.8. In embodiments, the crystalline Form A is characterized by a powder X-ray diffraction pattern that has a characteristic peak in degrees 2θ at about 9.8, is characterized by a powder X-ray diffraction pattern that has a characteristic peak in degrees 2θ at 9.8, 14.6, and 15.1, and/or is characterized by a powder X-ray diffraction pattern that has a characteristic peak in degrees 2θ at about 9.8, 14.6, 15.1, 19.7, and 24.3.

In some embodiments, the crystalline Form A is characterized by a powder X-ray diffraction pattern that has a characteristic peak in degrees 2θ at about 6.6, 9.8, 13.9, and 14.6, or is characterized by a powder X-ray diffraction pattern that has a characteristic peak in degrees 2θ at about 6.6, 9.8, 13.9, 14.6, 15.1, 16.8, 18.5, 19.7, 21.1, and 24.3.

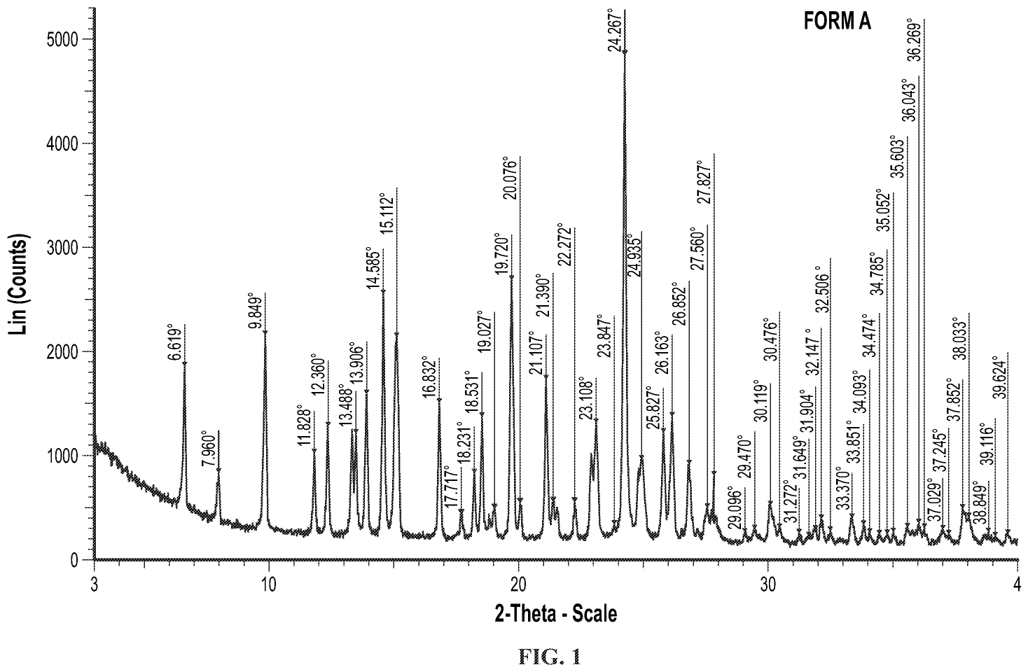

For example, a contemplated crystalline form has a powder X-ray diffraction pattern shown in FIG. 1. In one embodiment, the powder X-ray diffraction pattern of the crystalline form was obtained using Cu Kα radiation.

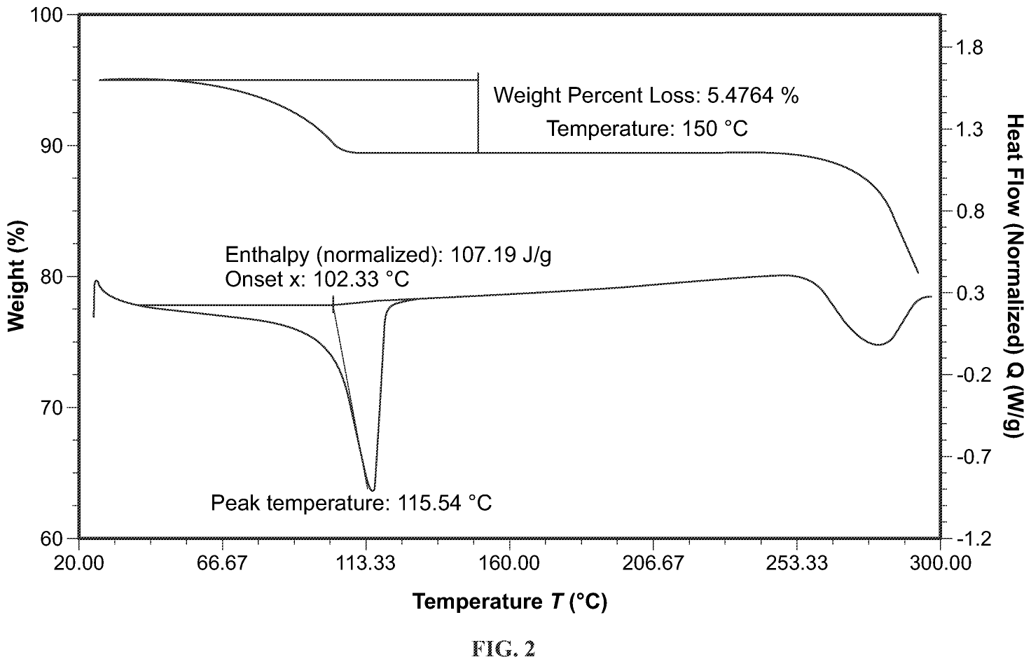

The contemplated crystalline Form A of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methanol solvate, may be characterized by a differential scanning calorimetry (DSC) profile profile showing a characteristic endotherm with an onset of about 102° C., and a peak of about 116° C. Form A, for example, may be characterized by the differential scanning calorimetry profile shown in FIG. 2.

The contemplated crystalline Form A of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methanol solvate, may be characterized by a thermogravimetric analysis (TGA) profile showing a mass loss of about 5.5 wt. % up to about 150° C. (FIG. 2).

### Form B

In certain embodiments, disclosed herein is a crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate (referred to herein as “Form B”), characterized by a powder X-ray diffraction pattern that has a characteristic peak in degrees 2θ at about 6.5. In embodiments, Form B is characterized by a powder X-ray diffraction pattern that has a characteristic peak in degrees 2θ at about 6.5 and 8.4, is characterized by a powder X-ray diffraction pattern that has a characteristic peak in degrees 2θ at about 6.5, 7.9, and 8.4, and/or is characterized by a powder X-ray diffraction pattern that has a characteristic peak in degrees 2θ at about 6.5, 7.9, 8.4, 13.9, 17.5, 20.3, and 24.2.

In embodiments, the crystalline Form B is characterized by a powder X-ray diffraction pattern that has a characteristic peak in degrees 2θ at about 6.5, 7.1, and 7.9, or is characterized by a powder X-ray diffraction pattern that has a characteristic peak in degrees 2θ at about 6.5, 7.1, 7.9, 8.4, 13.9, 14.7, 17.5, 20.3, 22.9, and 24.2.

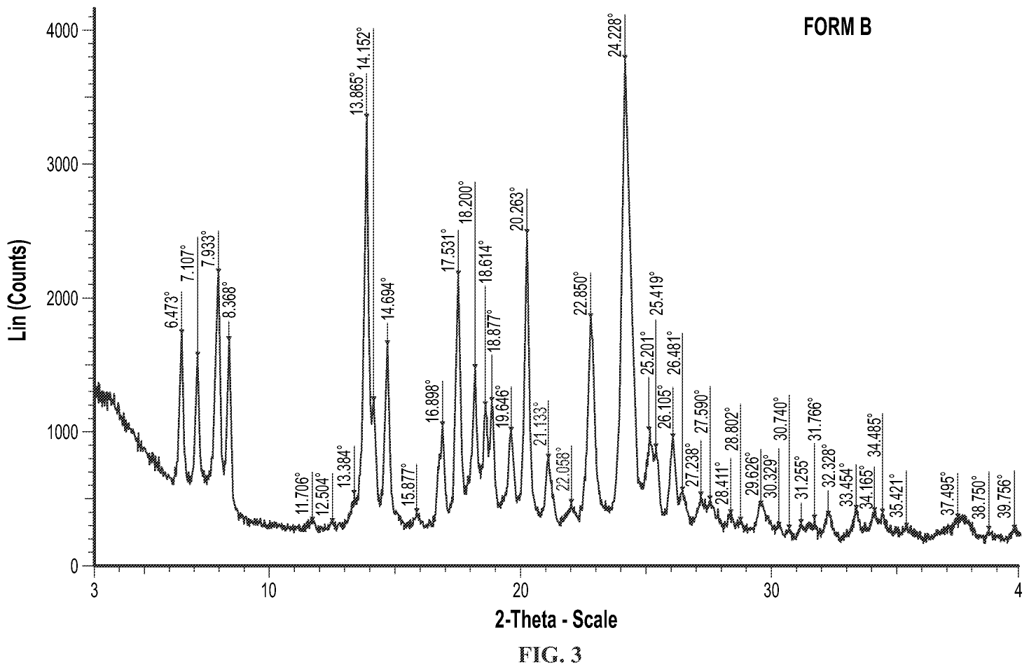

For example, a contemplated crystalline Form B has a powder X-ray diffraction pattern shown in FIG. 3. In one embodiment, the powder X-ray diffraction pattern of the crystalline form was obtained using Cu Kα radiation.

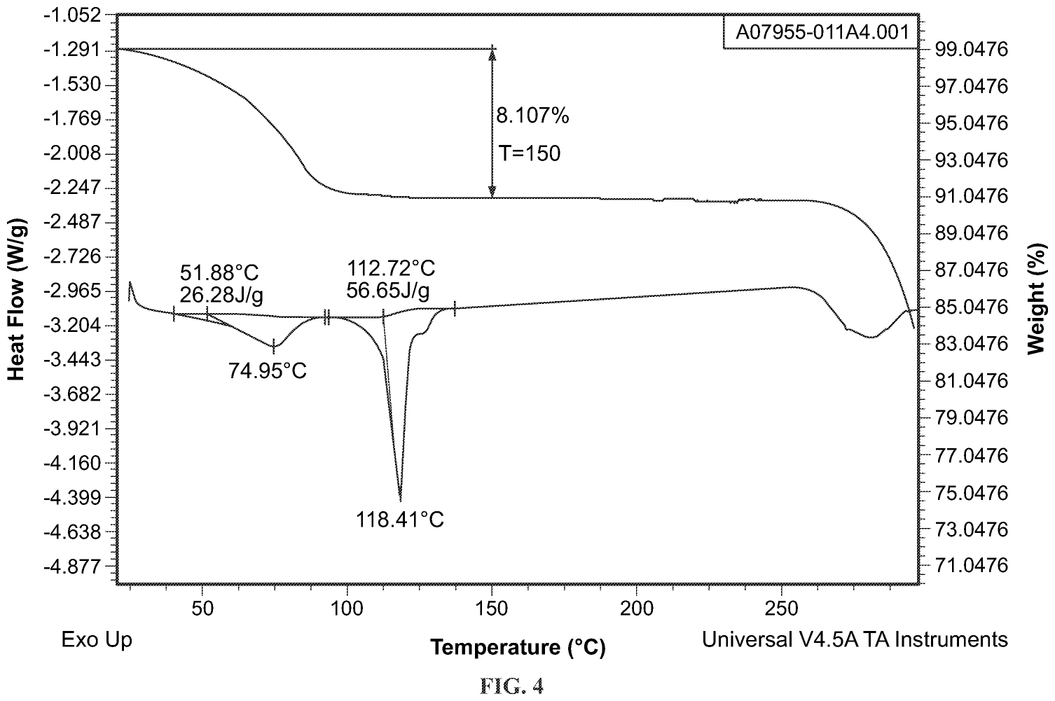

The contemplated crystalline Form B of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate, may be characterized by a differential scanning calorimetry (DSC) profile profile showing a characteristic endotherm with an onset of about 52° C., and a peak of about 118° C. Form B, for example, may be characterized by the differential scanning calorimetry profile shown in FIG. 4.

The contemplated crystalline Form B of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate, may be characterized by a thermogravimetric analysis (TGA) profile showing a mass loss of about 8.1 wt. % up to about 150° C. (FIG. 4).

### Form C

In certain embodiments, disclosed herein is a crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate (referred to herein as “Form C”), characterized by a powder X-ray diffraction pattern having a characteristic peak in degrees 2θ at about 3.3.

In one embodiment, the crystalline Form C of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate, is characterized by a powder X-ray diffraction pattern that has a characteristic peak in degrees 2θ at about 5.4, is characterized by a powder X-ray diffraction pattern that has a characteristic peak in degrees 2θ at about 3.3, 3.7, and 5.4, and/or is characterized by a powder X-ray diffraction pattern that has a characteristic peak in degrees 2θ at about 3.3, 3.7, 5.4, 7.0 and 7.2.

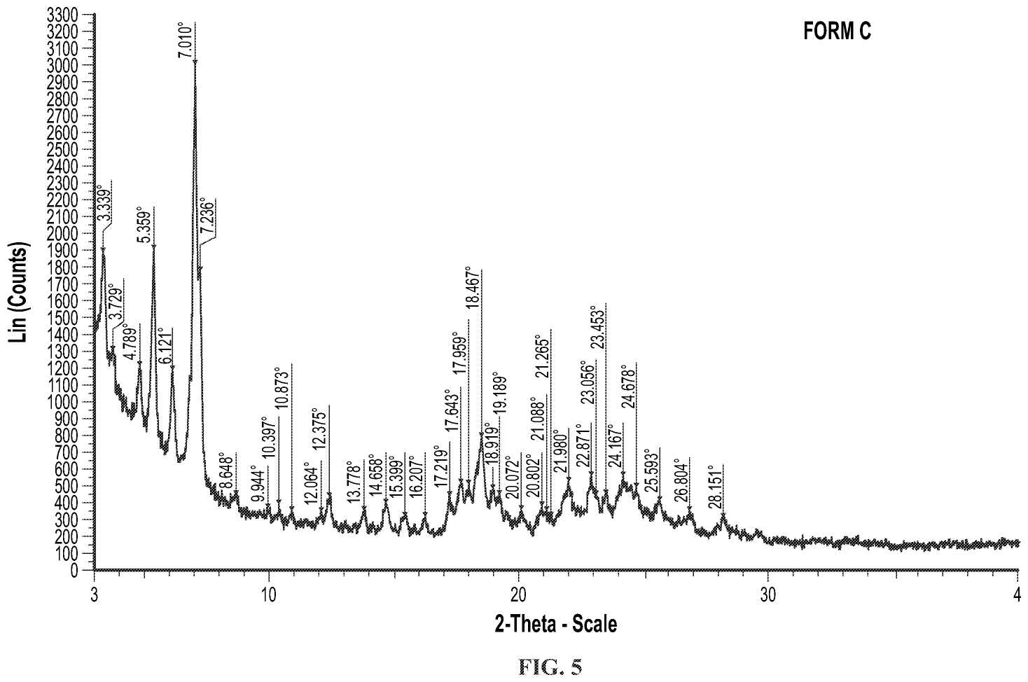

In some embodiments, the crystalline Form C is characterized by a powder X-ray diffraction pattern that has a characteristic peak in degrees 2θ at about 3.3, 3.7, 4.8, 5.4, and 6.1, is characterized by a powder X-ray diffraction pattern that has a characteristic peak in degrees 2θ at about 3.3, 3.7, and 4.8, or is characterized by a powder X-ray diffraction pattern that has a characteristic peak in degrees 2θ at about 3.3, 3.7, 4.8, 5.4, 6.1, 7.0, 7.2, 18.5, 22.9 and 24.2. For example, a contemplated crystalline form has a powder X-ray diffraction pattern shown in FIG. 5. In one embodiment, the powder X-ray diffraction pattern of the crystalline form was obtained using Cu Kα radiation.

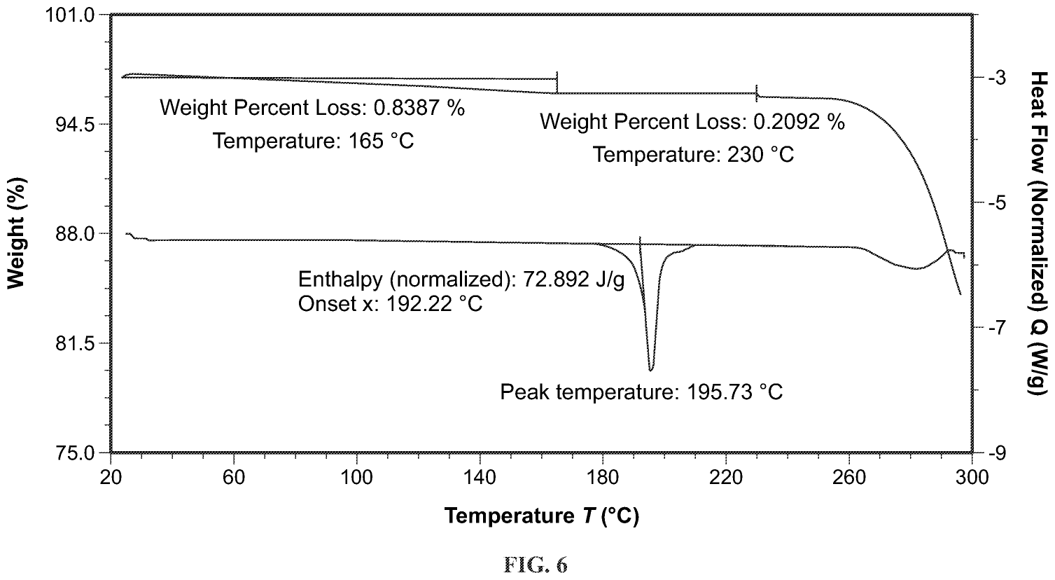

The contemplated crystalline Form C of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate, may be characterized by a differential scanning calorimetry (DSC) profile profile showing a characteristic endotherm with an onset of about 171 to about 193° C. (e.g., 192)° C., and a peak of about 180 to about 197° C. (e.g., 196° C.). Form C, for example, may be characterized by the differential scanning calorimetry profile shown in FIG. 6.

The contemplated crystalline Form C of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate, may be characterized by a thermogravimetric analysis (TGA) profile showing a mass loss of about 0.8 wt. % up to about 165° C. (FIG. 6). In some embodiments, the crystalline Form C may be characterized by a thermogravimetric analysis (TGA) profile showing a mass loss of about 0.2 wt. % up to about 165 to about 230° C. (FIG. 6).

### Form D

In certain embodiments, disclosed herein is a crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl ethyl ketone solvate (referred to herein as “Form D”), characterized by a powder X-ray diffraction pattern that has a characteristic peak in degrees 2θ at about 6.3. In embodiments, Form D is characterized by a powder X-ray diffraction pattern that has a characteristic peak in degrees 2θ at about 6.3 and 11.5, is characterized by a powder X-ray diffraction pattern that has a characteristic peak in degrees 2θ at about 6.3, 11.5, and 17.3, and/or is characterized by a powder X-ray diffraction pattern that has a characteristic peak in degrees 2θ at about 6.3, 11.5, 17.3, 18.2, and 25.2.

In some embodiments, Form D is characterized by a powder X-ray diffraction pattern that has a characteristic peak in degrees 2θ at 6.3, 7.0, and 11.5, or is characterized by a powder X-ray diffraction pattern that has a characteristic peak in degrees 2θ at 6.3, 7.0, 11.5, 12.6, 14.2, 17.3, 18.2, 18.8, 19.2, and 25.2.

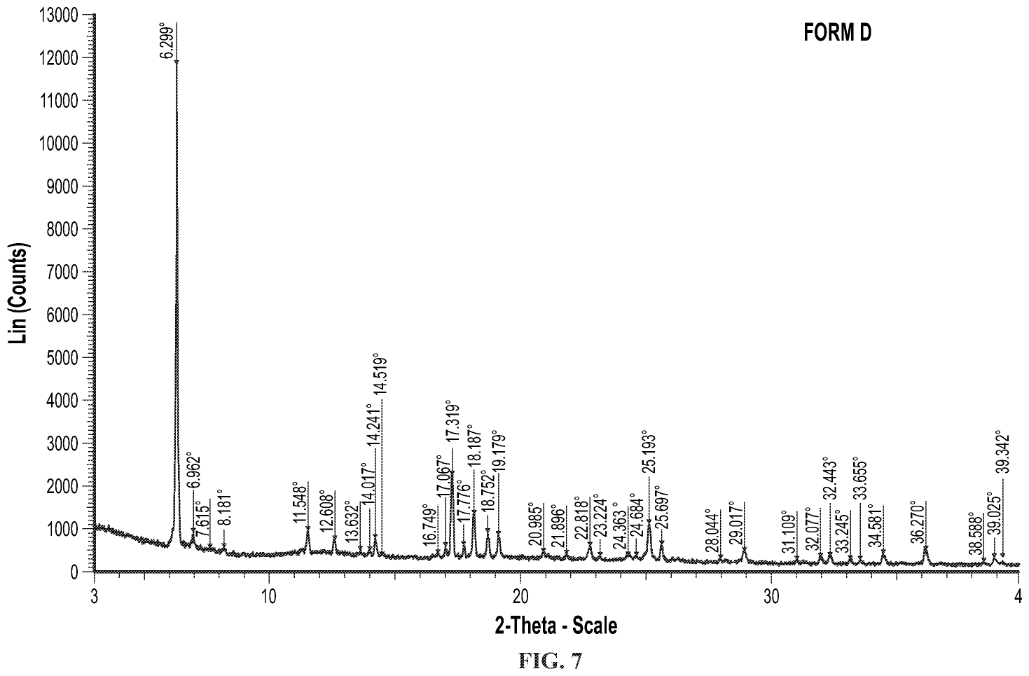

For example, a contemplated crystalline form has a powder X-ray diffraction pattern shown in FIG. 7. In one embodiment, the powder X-ray diffraction pattern of the crystalline form was obtained using Cu Kα radiation.

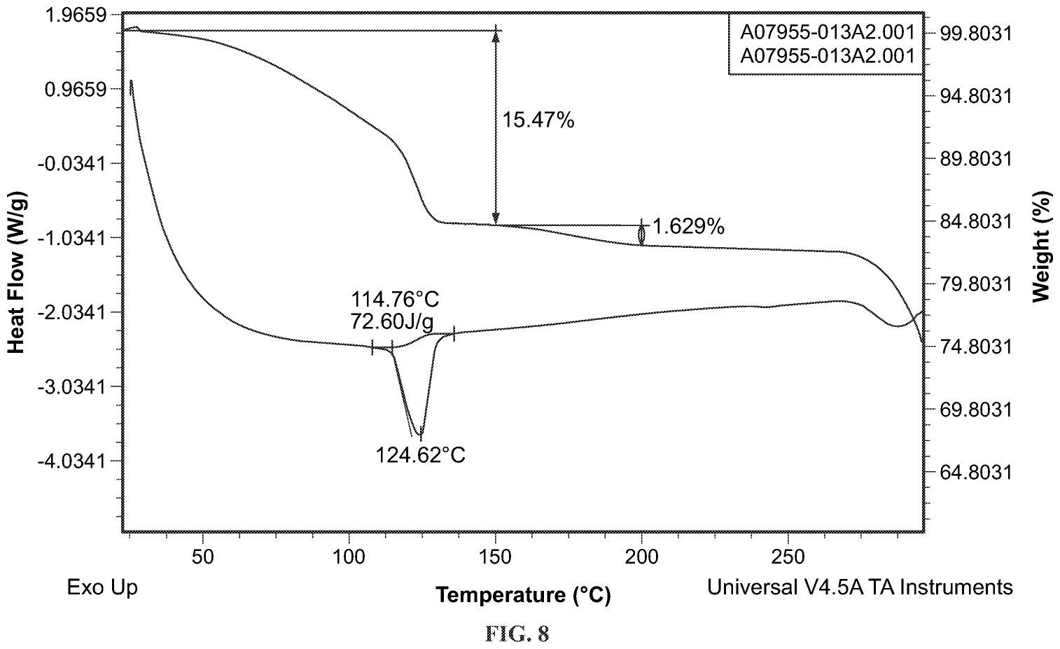

The contemplated crystalline Form D of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl ethyl ketone solvate, may be characterized by a differential scanning calorimetry (DSC) profile profile showing a characteristic endotherm with an onset of about 115° C., and a peak of about 125° C. Form D, for example, may be characterized by the differential scanning calorimetry profile shown in FIG. 8.

The contemplated crystalline Form D of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl ethyl ketone solvate, may be characterized by a thermogravimetric analysis (TGA) profile showing a mass loss of about 8.1 wt. % up to about 150° C. (FIG. 4).

### Form E

In certain embodiments, disclosed herein is a crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl isobutyl ketone solvate (referred to herein as “Form E”), characterized by a powder X-ray diffraction pattern that has a characteristic peak in degrees 2θ at about 6.2. In embodiments, Form E is characterized by a powder X-ray diffraction pattern that has a characteristic peak in degrees 2θ at about 12.9, is characterized by a powder X-ray diffraction pattern that has a characteristic peak in degrees 2θ at about 6.2, 12.9, and 14.7, and/or is characterized by a powder X-ray diffraction pattern that has a characteristic peak in degrees 2θ at about 6.2, 12.9, 14.7, 14.9, and 19.8.

In some embodiments, Form E is characterized by a powder X-ray diffraction pattern that has a characteristic peak in degrees 2θ at about 6.2, 12.5, 12.9, 13.7, 14.7, 14.9, 17.0, 19.0, 19.8, and 38.4

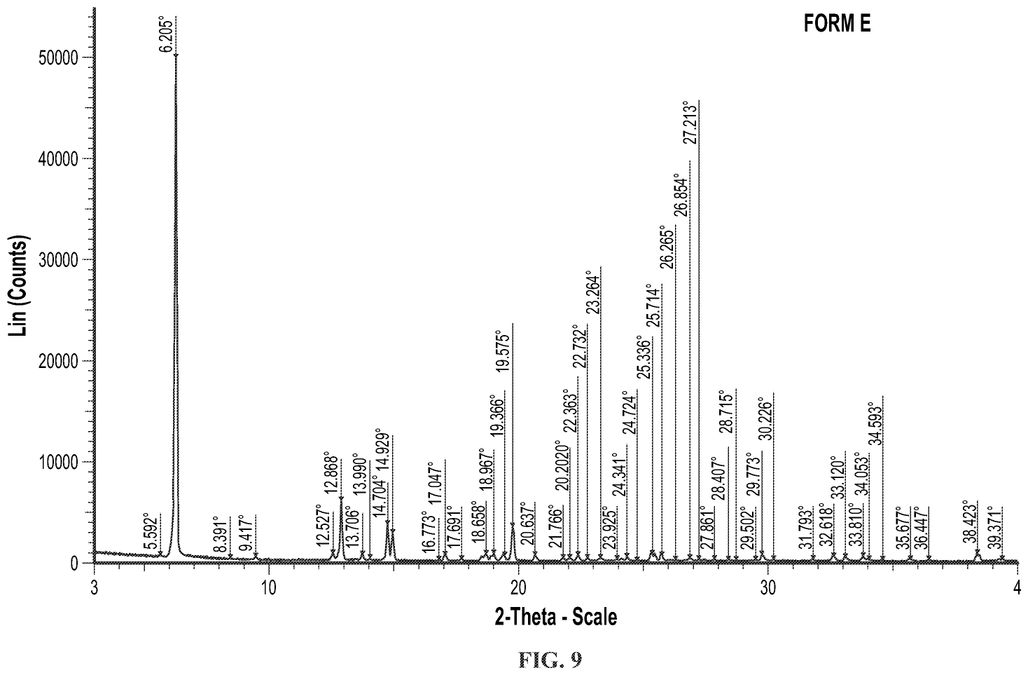

For example, a contemplated crystalline form has a powder X-ray diffraction pattern shown in FIG. 9. In one embodiment, the powder X-ray diffraction pattern of the crystalline form was obtained using Cu Kα radiation.

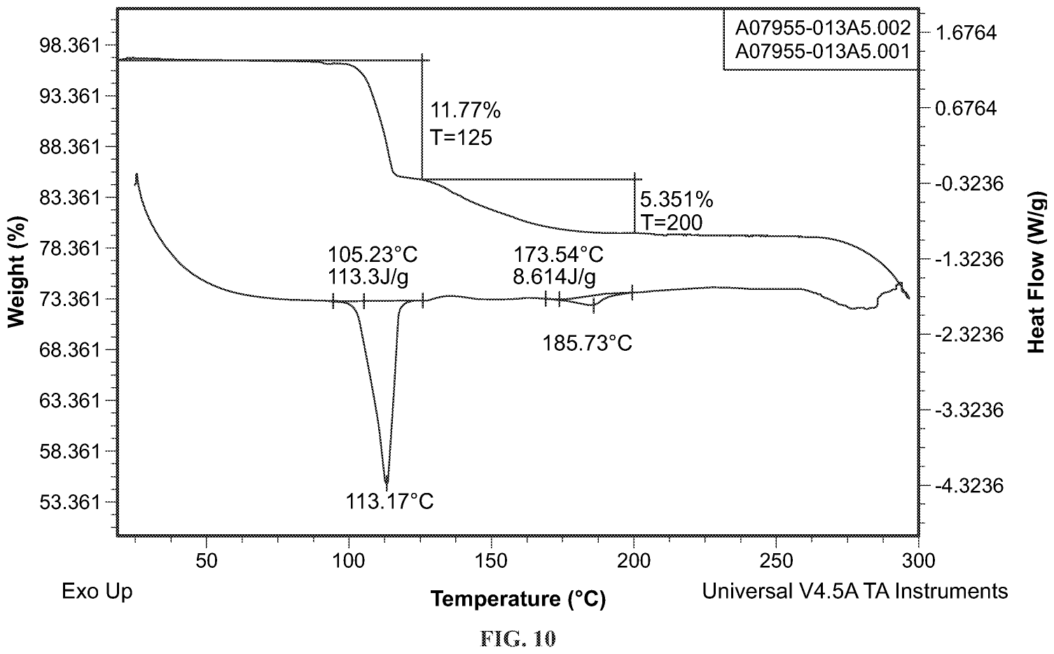

The contemplated crystalline Form E of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl isobutyl ketone solvate, may be characterized by a differential scanning calorimetry (DSC) profile profile showing a characteristic endotherm with an onset of about 105° C., and a peak of about 113° C. In embodiments, Form E may be characterized by a differential scanning calorimetry (DSC) profile profile showing a characteristic endotherm with an onset of about 174° C., and a peak of about 186° C. Form E, for example, may be characterized by the differential scanning calorimetry profile shown in FIG. 10.

The contemplated crystalline Form E of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl isobutyl ketone solvate, may be characterized by a thermogravimetric analysis (TGA) profile showing a mass loss of about 11.8 wt. % up to about 80 to about 125° C. (FIG. 10). Form E may be characterized by a thermogravimetric analysis (TGA) profile showing a mass loss of about 5.4 wt. % up to about 125 to about 200° C. (FIG. 10).

### Form F

In certain embodiments, disclosed herein is a crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate (referred to herein as “Form F”) characterized by a powder X-ray diffraction pattern that has a characteristic peak in degrees 2θ at about 8.2.

In one embodiment, the crystalline Form F of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate, is characterized by a powder X-ray diffraction pattern that has a characteristic peak in degrees 2θ at about 7.8, 8.2, and 13.7, and/or is characterized by a powder X-ray diffraction pattern that has a characteristic peak in degrees 2θ at about 7.8, 8.2, 13.7, 14.6, 20.1, and 24.1.

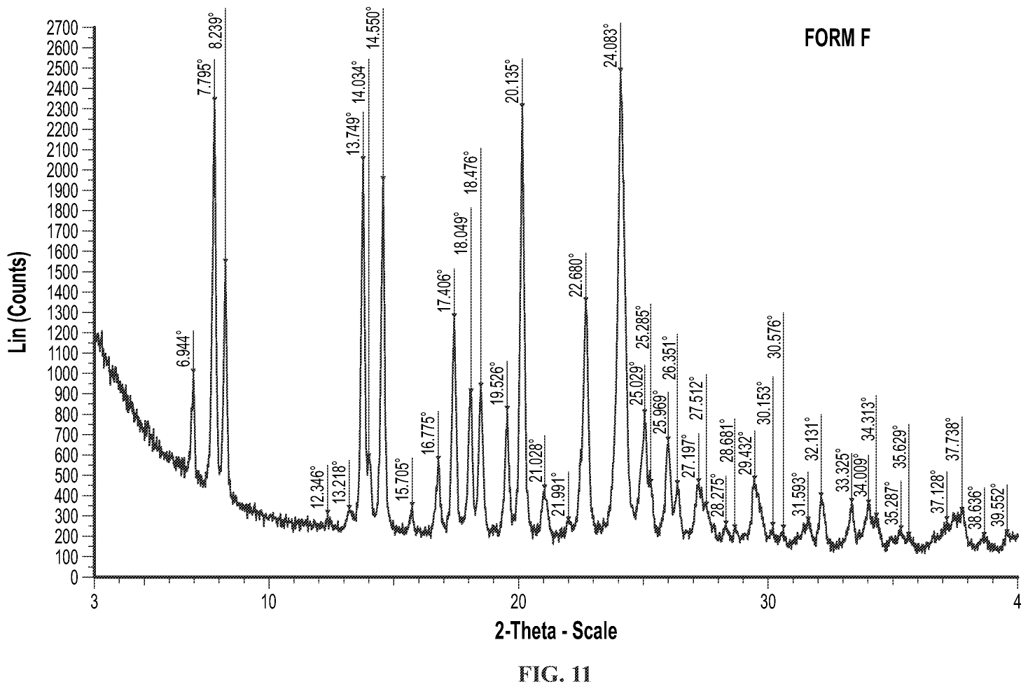

In some embodiments, the crystalline Form F is characterized by a powder X-ray diffraction pattern that has a characteristic peak in degrees 2θ at about 6.9, 7.8, 8.2, and 13.7, or is characterized by a powder X-ray diffraction pattern that has a characteristic peak in degrees 2θ at about 6.9, 7.8, 8.2, 13.7, 14.6, 17.4, 18.5, 20.1, 22.7, and 24.1. For example, a contemplated crystalline form has a powder X-ray diffraction pattern shown in FIG. 11. In one embodiment, the powder X-ray diffraction pattern of the crystalline form was obtained using Cu Kα radiation.

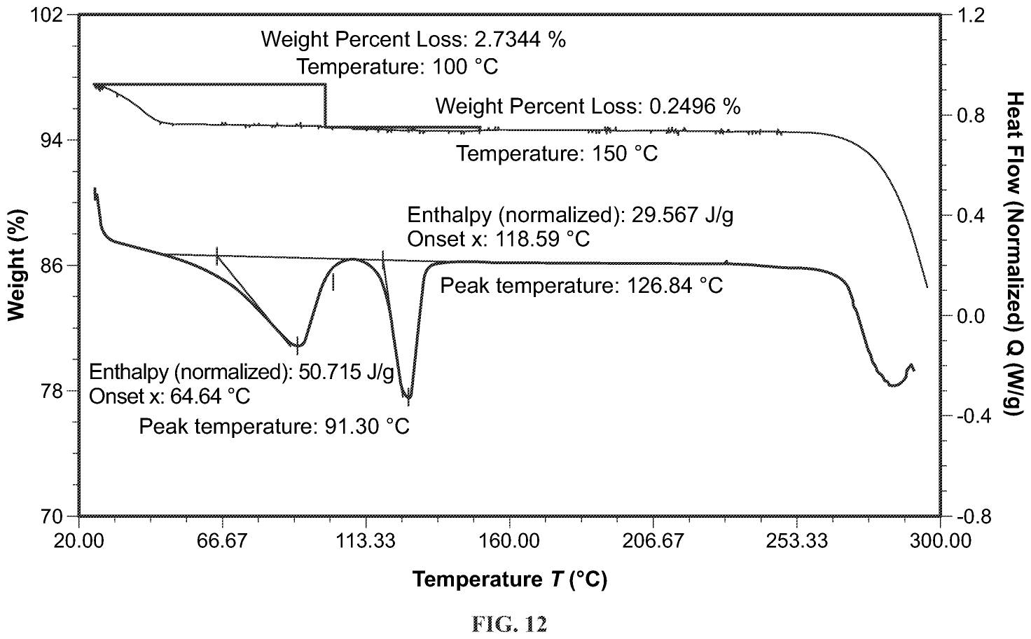

The contemplated crystalline Form F of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate, may be characterized by a differential scanning calorimetry (DSC) profile profile showing a characteristic endotherm with an onset of about 51 to about 65° C. (e.g., 65° C.), and a peak of about 79 to about 91° C. (e.g., 91° C.). In some embodiments, Form F may be characterized by a differential scanning calorimetry (DSC) profile profile showing a characteristic endotherm with an onset of about 117 to about 119° C. (e.g., 119° C.), and a peak of about 127 to about 128° C. (e.g., 127° C.). Form F, for example, may be characterized by the differential scanning calorimetry profile shown in FIG. 12.

The contemplated crystalline Form F of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate, may be characterized by a thermogravimetric analysis (TGA) profile showing a mass loss of about 2.7 wt. % up to about 100° C. (FIG. 12). In some embodiments, Form F may be characterized by a thermogravimetric analysis (TGA) profile showing a mass loss of about 0.2 wt. % up to about 100 to about 150° C. (FIG. 12).

In another embodiment, a substantially amorphous form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide is disclosed herein.

In a further embodiment, a pharmaceutical composition comprising a disclosed crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide and a pharmaceutically acceptable excipient is disclosed herein.

A crystalline described herein, e.g., 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate, can be formulated as a pharmaceutical composition using a pharmaceutically acceptable carrier and administered by a variety of routes. In some embodiments, such compositions are for oral administration. In some embodiments, compositions formulated for oral administration are provided as tablets. In some embodiments, such compositions are for parenteral (by injection) administration. In some embodiments, such compositions are for transdermal administration. In some embodiments, such compositions are for topical administration. In some embodiments, such compositions are for intravenous (IV) administration. In some embodiments, such compositions are for intramuscular (IM) administration. Such pharmaceutical compositions and processes for preparing them are well known in the art. Sec, e.g., REMINGTON: THE SCIENCE AND PRACTICE OF PHARMACY (A. Gennaro, et al., eds., 19^{th} ed., Mack Publishing Co., 1995).

### Method for Making

The disclosure also provides a method for making the crystalline Form C, the method comprising contacting an amorphous form of a compound of Formula I with a solvent to form a solubilized form or gel; stirring the solubilized form or gel; and drying the solubilized form or gel to form the crystalline form; wherein the compound of Formula I is 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide.

In another part of the disclosure, provided herein is a method for making the crystalline Form C, the method comprising contacting an amorphous form, a solvate form, a hydrate form, or a mixture thereof of a compound of Formula I with a solvent to form a solubilized form, a gel or a solid; stirring the solubilized form, the gel, or the solid; and drying the solubilized form, the gel, or the solid to form the crystalline form; wherein the compound of Formula I is 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide.

In some embodiments, the solvate form is an acetone solvate. In some embodiments, In some embodiments, the solvent comprises isopropanol. In some embodiments, the solvent is isopropanol. In some embodiments, the solvent comprises isopropanol and water. In some embodiments, the solvent is isopropanol and water. In some embodiments, the solvent comprises water. In some embodiments, the solvent is water. In some embodiments, the stirring is performed at a temperature of about 25-40° C. (e.g., 20° C., 25° C., 30° C., 35° C., 40° C., and 45)° C. In some embodiments, the stirring is performed at a temperature of about 40-60° C. (e.g., 35° C., 40° C., 45° C., 50° C., 55° C., 60° C., and 65° C.). In some embodiments, the stirring is performed at a temperature of about 60-80° C. (e.g., 55° C., 60° C., 65° C., 70° C., 75° C., 80° C., and 85° C.). In some embodiments, the stirring is performed at about 25-80° C. (e.g., 20° C., 25° C., 30° C., 35° C., 40° C., 45° C., 50° C., 55° C., 60° C., 65° C., 70° C., 75° C., 80° C., and 85° C.). In some embodiments, drying comprises drying under vacuum. In some embodiments, drying comprises drying under reduced pressure.

In another embodiments, provided herein is a method for making Form F, the method comprising contacting an amorphous form of a compound of Formula I with a solvent comprising methylethylketone to form a suspension; stirring the suspension; drying the suspension to obtain the crystalline form; wherein the compound of Formula I is 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide. In some embodiments, the solvent further comprises water. In some embodiments, the stirring is performed at room temperature.

### II. Methods of Use and Treatment

In some embodiments, the disclosure provides a method of treating a viral infection in a patient in need thereof, comprising administering to the patient an effective amount of a disclosed crystalline compound, for example, a disclosed crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide. In other embodiments, the disclosure provides a method of treating a viral infection in a patient in need thereof, comprising administering to the patient an effective amount of a pharmaceutical composition comprising a disclosed crystalline compound, for example, a disclosed crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide.

In certain embodiments, the disclosure provides a method of ameliorating or treating a viral infection in a patient in need thereof, comprising administering to the patient a therapeutically effective amount of any of the crystalline compounds described herein (e.g., Form A, B, C, D, E, or F). In some embodiments, the viral infection is from a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebola virus, a morbillivirus, an enterovirus (e.g., enterovirus 71 (EV71), an orthopneumovirus, a lentivirus, arenavirus, a herpes virus, and a hepatovirus. In certain embodiments, the viral infection is a coronavirus infection. In some embodiments, the viral infection is a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19). In embodiments, the viral infection is SARS-CoV-2.

In some embodiments, the viral infection is from a virus selected from the group consisting of calicivimses, MD145, murine norovirus, vesicular exanthema of swine virus, abbit hemorrhagic disease virus, porcine teschovirus, bovine coronavirus, feline infectious peritonitis virus, EV-68 virus, EV-71 virus, poliovirus, norovirus, human rhinovirus (HRV), hepatitis A virus (HAV) and foot-and-mouth disease virus (FMDV).

In embodiments, the viral infection is an arenavirus infection. In some embodiments, the arenavirus is selected from the group consisting of: Junin virus, Lassa virus, Lujo virus, Machupo virus, and Sabia virus. In some embodiments, the viral infection is an influenza infection. In some embodiments, the influenza is influenza H1N1, H3N2 or H5N1.

Another aspect of the disclosure provides methods of treating patients suffering from a viral infection, e.g., a noroviral infection. In some embodiments, the disclosure provides a method of treating a viral infection from a norovirus in a patient in need thereof, comprising administering to the patient a therapeutically effective amount of any of the crystalline compounds described herein.

Also provided herein, in certain embodiments, is a method of inhibiting transmission of a virus, a method of inhibiting viral replication, a method of minimizing expression of viral proteins, or a method of inhibiting virus release, comprising administering a therapeutically effective amount of a crystalline compound described herein to a patient suffering from the virus, and/or contacting an effective amount of a crystalline compound described herein with a virally infected cell. In some embodiments, the method further comprises administering another therapeutic. In some embodiments, the method further comprises administering an additional anti-viral therapeutic. In embodiments, the anti-viral therapeutic is selected from the group consisting of ribavirin, favipiravir, ST-193, oseltamivir, zanamivir, peramivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ccoliever, epigallocatechin gallate, ctravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine. In some embodiments, the another therapeutic is selected from the group consisting of protease inhibitors, fusion inhibitors, M2 proton channel blockers, polymerase inhibitors, 6-endonuclease inhibitors, neuraminidase inhibitors, reverse transcriptase inhibitor, aciclovir, acyclovir, protease inhibitors, arbidol, atazanavir, atripla, boceprevir, cidofovir, combivir, darunavir, docosanol, edoxudine, entry inhibitors, entecavir, famciclovir, fomivirsen, fosamprenavir, foscarnet, fosfonet, ganciclovir, ibacitabine, immunovir, idoxuridine, imiquimod, inosine, integrase inhibitor, interferons, lopinavir, loviride, moroxydine, nexavir, nucleoside analogues, penciclovir, pleconaril, ipodophyllotoxin, ribavirin, tipranavir, trifluridine, trizivir, tromantadine, truvada, valaciclovir, valganciclovir, vicriviroc, vidarabine, viramidine, and zodovudine. In embodiments, the additional anti-viral therapeutic is selected from the group consisting of lamivudine, an interferon alpha, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), rifampicin, zanamivir, oseltamivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, ctravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine.

Contemplated patients include not only humans, but other animals such as companion animals (e.g. dogs, cats), domestic animals (e.g. cow, swine), and wild animals (e.g. monkeys, bats, snakes).

Accordingly, in one embodiment, described herein is a method of ameliorating or treating a viral infection in a patient in need thereof, comprising administering to the patient a therapeutically effective amount of a crystalline compound described herein (e.g., Form A, B, C, D, E, or F).

Other contemplated methods of treatment include a method of treating or ameliorating a virus infection condition or co-morbidity, by administering an effective amount a crystalline compound disclosed herein to a subject in need thereof.

Exemplary co-morbidities include lung diseases, cardiac disorders, endocrine disorders, respiratory disorders, hepatic disorders, skeletal disorders, psychiatric disorders, metabolic disorders, and reproductive disorders.

In some embodiments, the viral infection is from a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, arenavirus, a herpes virus, and a hepatovirus. In some embodiments, the viral infection is a coronavirus infection. In some embodiments, the viral infection is a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19). In some embodiments, the viral infection is SARS-CoV-2. In some embodiments, the viral infection is an arenavirus infection. In some embodiments, the arenavirus is selected from the group consisting of: Junin virus, Lassa virus, Lujo virus, Machupo virus, and Sabia virus. In some embodiments, the viral infection is an influenza infection. In some embodiments, the influenza is influenza H1N1, H3N2 or H5N1. In some embodiments, the viral infection is a respiratory viral infection. In some embodiments, the viral infection is an upper respiratory viral infection or a lower respiratory viral infection. In some embodiments, the method further comprises administering another therapeutic.

In certain embodiments, the virus is selected from the group consisting of a retrovirus (e.g., human immunodeficiency virus (HIV), simian immunodeficiency virus (SIV), human T-cell lymphotropic virus (HTLV)-1, HTLV-2, HTLV-3, HTLV-4), Ebola virus, hepatitis A virus, hepatitis B virus, hepatitis C virus, a herpes simplex virus (HSV) (e.g., HSV-1, HSV-2, varicella zoster virus, cytomegalovirus), an adenovirus, an orthomyxovirus (e.g., influenza virus A, influenza virus B, influenza virus C, influenza virus D, togavirus), a flavivirus (e.g., dengue virus, Zika virus), West Nile virus, Rift Valley fever virus, an arenavirus, Crimean-Congo hemorrhagic fever virus, an echovirus, a rhinovirus, coxsackie virus, a coronavirus (e.g., Severe acute respiratory syndrome coronavirus 2 (SARS-CoV-2), coronavirus disease 2019 (COVID-19), a respiratory syncytial virus, a mumps virus, a rotavirus, measles virus, rubella virus, a parvovirus (e.g., an adeno-associated virus), a vaccinia virus, a variola virus, a molluscum virus, bovine leukemia virus, bovine diarrhea virus, a poliovirus, St. Louis encephalitis virus, Japanese encephalitis virus, a tick-borne encephalitis virus, Murray Valley virus, Powassan virus, Rocio virus, louping-ill virus, Banzi virus, Ilheus virus, Kokobera virus, Kunjin virus, Alfuy virus, a rabies virus, a polyomavirus (e.g., JC virus, BK virus), an alphavirus, and a rubivirus (e.g., rubella virus).

In certain embodiments, the disease or disorder is a viral infection, e.g., a disease or disorder selected from the group consisting of acquired immune deficiency syndrome (AIDS), HTLV-1 associated myelopathy/tropical spastic paraparesis, Ebola virus disease, hepatitis A, hepatitis B, hepatitis C, herpes, herpes zoster, acute varicella, mononucleosis, respiratory infections, pneumonia, influenza, dengue fever, encephalitis (e.g., Japanese encephalitis, St. Louis encephalitis, or tick-borne encephalitis such as Powassan encephalitis), West Nile fever, Rift Valley fever, Crimean-Congo hemorrhagic fever, Kyasanur Forest disease, Yellow fever, Zika fever, aseptic meningitis, myocarditis, common cold, lung infections, molloscum contagiosum, enzootic bovine leucosis, coronavirus disease 2019 (COVID-19), mumps, gastroenteritis, measles, rubella, slapped-check disease, smallpox, warts (e.g., genital warts), molluscum contagiosum, polio, rabies, and Pityriasis rosea.

In some embodiments, the virus is an RNA virus (having a genome that is composed of RNA). RNA viruses may be single-stranded RNA (ssRNA) or double-stranded RNA (dsRNA). RNA viruses have high mutation rates compared to DNA viruses, as RNA polymerase lacks proofreading capability (see, e.g., Steinhauer D A, Holland J J (1987). “Rapid evolution of RNA viruses”. Annu. Rev. Microbiol. 41: 409-33). In some embodiments, the RNA virus is a positive-strand RNA virus (e.g., a SARS-CoV virus, polio virus, Coxsackie virus, Enterovirus, Human rhinovirus, Foot/Mouth disease virus, encephalomyocarditis virus, Dengue virus, Zika virus, Hepatitis C virus, or New Castle Disease virus).

RNA viruses are classified by the type of genome (double-stranded, negative (−), or positive (+) single-stranded). Double-stranded RNA viruses contain a number of different RNA molecules, each coding for one or more viral proteins. Positive-sense ssRNA viruses utilize their genome directly as mRNA; ribosomes within the host cell translate mRNA into a single protein that is then modified to form the various proteins needed for viral replication. One such protein is RNA-dependent RNA polymerase (RNA replicase), which copies the viral RNA in order to form a double-stranded, replicative form. Negative-sense ssRNA viruses have their genome copied by an RNA replicase enzyme to produce positive-sense RNA for replication. Therefore, the virus comprises an RNA replicase enzyme. The resultant positive-sense RNA then acts as viral mRNA and is translated by the host ribosomes. In some embodiments, the virus is a dsRNA virus. In some embodiments, the virus is a negative ssRNA virus. In some embodiments, the virus is a positive ssRNA virus. In some embodiments, the positive ssRNA virus is a coronavirus.

SARS-CoV2, also sometimes referred to as the novel coronavirus of 2019 or 2019-nCoV, is a positive-sense single-stranded RNA virus. SARS-CoV-2 has four structural proteins, known as the S (spike), E (envelope), M (membrane), and N (nucleocapsid) proteins. The N protein holds the RNA genome together; the S, E, and M proteins form the viral envelope. Spike allows the virus to attach to the membrane of a host cell, such as the ACE2 receptor in human cells (Kruse R. L. (2020), Therapeutic strategies in an outbreak scenario to treat the novel coronavirus originating in Wuhan, China (version 2). F1000Research, 9:72). SARS-CoV2 is the highly contagious, causative viral agent of coronavirus disease 2019 (COVID19), a global pandemic.

In some embodiments, the virus is a DNA virus (having a genome that is composed of DNA). Exemplary DNA viruses include, without limitation, parvoviruses (e.g., adeno-associated viruses), adenoviruses, asfarviruses, herpesviruses (e.g., herpes simplex virus 1 and 2 (HSV-1 and HSV-2), Epstein-Barr virus (EBV), cytomegalovirus (CMV)), papillomaviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). Exemplary RNA viruses include, without limitation, bunyaviruses (e.g., hantavirus), coronaviruses, flaviviruses (e.g., yellow fever virus, west Nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, calicivirus, noroviruses (e.g., Norwalk virus), poliovirus, respiratory syncytial virus (RSV), retroviruses (e.g., human immunodeficiency virus-1 (HIV-1)) and toroviruses.

The methods described herein may inhibit viral replication transmission, replication, assembly, or release, or minimize expression of viral proteins. In one embodiment, described herein is a method of inhibiting transmission of a virus, a method of inhibiting viral replication, a method of minimizing expression of viral proteins, or a method of inhibiting virus release, comprising administering a therapeutically effective amount of a crystalline compound described herein, or a pharmaceutically acceptable salt thereof, to a patient suffering from the virus, and/or contacting an effective amount of a crystalline compound described herein, with a virally infected cell.

Also described herein is a method of treating a respiratory disorder in a subject in need thereof, comprising administering to the patient a therapeutically effective amount of a crystalline compound (e.g., Form A, B, C, D, E, or F) described herein. In certain embodiments, the respiratory disorder is selected from the group consisting of chronic obstructive pulmonary disease (COPD), asthma, fibrosis, chronic asthma, acute asthma, lung disease secondary to environmental exposures, acute lung infection, chronic lung infection, al antitrypsin disease, cystic fibrosis and an autoimmune disease. In some embodiments, the respiratory disorder is associated with a heart attack.

Also described herein is a method of treating a disorder associated with cathepsin (e.g. Cathepsin K) in a subject in need thereof, comprising administering to the patient a therapeutically effective amount of a crystalline compound described herein (e.g., Form A, B, C, D, E, or F). In some embodiments, the disorder is a cathepsin dependent condition or disease. In embodiments, the disorder is selected from the group consisting of breast cancer, pycnodysostosis, glioblastoma, osteosclerosis, osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, atherosclerosis, obesity, glaucoma, chronic obstructive pulmonary disease, metastatic bone disease, hypercalcemia of malignancy, and multiple myeloma.

Crystalline compounds described herein, e.g., Form A, B, C, D, E, F, etc as defined herein, can be administered in combination with one or more additional therapeutic agents to treat a disorder described herein, such as an infection by a pathogen described herein, e.g., a virus, fungus, or protozoan. For clarity, contemplated herein are both a fixed composition comprising a disclosed crystalline compound and another therapeutic agent such as disclosed herein, and methods of administering, separately a disclosed crystalline compound and a disclosed therapeutic. For example, provided in the present disclosure is a pharmaceutical composition comprising a crystalline compound described herein, e.g., a crystalline Form C as defined herein, one or more additional therapeutic agents, and a pharmaceutically acceptable excipient. In some embodiments, a crystalline Form C as defined herein and one additional therapeutic agent is administered. In some embodiments, a disclosed crystalline compound as defined herein and two additional therapeutic agents are administered. In some embodiments, a disclosed crystalline compound as defined herein and three additional therapeutic agents are administered. Combination therapy can be achieved by administering two or more therapeutic agents, each of which is formulated and administered separately. For example, a crystalline compound of e.g. Form A, B, C, D, E, and F, as defined herein and an additional therapeutic agent can be formulated and administered separately. Combination therapy can also be achieved by administering two or more therapeutic agents in a single formulation, for example a pharmaceutical composition comprising a crystalline compound Form C as one therapeutic agent and one or more additional therapeutic agents such as an antibiotic, a viral protease inhibitor, or an anti-viral nucleoside anti-metabolite. For example, a crystalline Form C as defined herein and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

In some embodiments, the one or more additional therapeutic agents that may be administered in combination with a crystalline compound provided herein can be an antibiotic, a viral protease inhibitor, an anti-viral anti-metabolite, a lysosomotropic agent, a M2 proton channel blocker, a polymerase inhibitor (e.g., EIDD-2801, which is also known as MOLNUPIRAVIR), aneuraminidase inhibitor, a reverse transcriptase inhibitor, a viral entry inhibitor, an integrase inhibitor, interferons (e.g., types I, II, and III), or a nucleoside analogue. In some embodiments, the one or more additional therapeutic agents that may be administered in combination with crystalline compounds provided herein can be a steroid (e.g., corticosteroids, such as bethamethasone, prednisone, prednisolone, triamcinolone, methylprednisolone, dexamethasone; mineralcorticoid such as fludrocortisone; glucocorticoids, such as hydrocortisone, cortisone, ethamethasoneb, prednisone, prednisolone, triamcinolone, dexamethasone; vitamin D such as dihydrotachysterol; androgens such as apoptone, oxandrolone, oxabolone, testosterone, nandrolone (also known as anabolic steroids), oestrogens such as diethylstilbestrol, progestins such as danazol, norethindrone, medroxyprogesterone acetate, 17-Hydroxyprogesterone caproate; and progestins such as mifepristone and gestrinone) or an immunomodulator (e.g., 6Mercaptopurine, 6MP, Alferon N, anakinra, Arcalyst, Avonex, AVOSTARTGRIP, Bafiertam, Berinert, Betaseron, BG-12, C1 esterase inhibitor recombinant, C1 inhibitor human, Cinryze, Copaxone, dimethyl fumarate, diroximel fumarate, ecallantide, emapalumab, emapalumab-lzsg, Extavia, fingolimod, Firazyr, Gamifant, Gilenya, glatiramer, Glatopa, Haegarda, icatibant, Infergen, interferon alfa n3, interferon alfacon 1, interferon beta 1a, interferon beta 1b, Kalbitor, Kineret, mercaptopurine, monomethyl fumarate, peginterferon beta-1a, Plegridy, Purinethol, Purixan, Rebif, Rebif Rebidose, remestemcel-L, rilonacept, ropeginterferon alfa 2b, Ruconest, Ryoncil, siltuximab, sutimlimab, Sylvant, Tecfidera, and Vumerity). In some embodiments, the one or more additional therapeutic agent is Cathepsin L. In some embodiments, the one or more additional therapeutic agent is dehydrodidemnin B (also known as Plitidepsin or APLIDIN) or Zotatifin (eFT226).

In some embodiments, methods described herein further comprise administering an additional anti-viral therapeutic. In some embodiments, the anti-viral therapeutic is selected from the group consisting of ribavirin, favipiravir, ST-193, oseltamivir, zanamivir, peramivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ccoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine. In some embodiments, the another therapeutic is selected from the group consisting of protease inhibitors (e.g., nafamostat, camostat, gabexate, epsilon-aminocaproic acid and aprotinin), fusion inhibitors (e.g., BMY-27709, CL 61917, and CL 62554), M2 proton channel blockers (e.g., amantadine and rimantadine), polymerase inhibitors (e.g., 2-deoxy-2′fluoroguanosides (2′-fluoroGuo), 6-endonuclease inhibitors (e.g., L-735,822 and flutamide) neuraminidase inhibitors (e.g., zanamivir (Relenza), oseltamivir, peramivir and ABT-675 (A-315675), reverse transcriptase inhibitor (e.g., abacavir, adefovir, delavirdine, didanosine, efavirenz, emtricitabine, lamivudine, nevirapine, stavudine, tenofovir, tenofovir disoproxil, and zalcitabine), acyclovir, acyclovir, protease inhibitors (e.g., amprenavir, indinavir, nelfinavir, ritonavir, and saquinavir), arbidol, atazanavir, atripla, boceprevir, cidofovir, combivir, darunavir, docosanol, edoxudine, entry inhibitors (e.g., enfuvirtide and maraviroc), entecavir, famciclovir, fomivirsen, fosamprenavir, foscarnet, fosfonet, ganciclovir, ibacitabine, immunovir, idoxuridine, imiquimod, inosine, integrase inhibitor (e.g., raltegravir), interferons (e.g., types I, II, and III), lopinavir, loviride, moroxydine, nexavir, nucleoside analogues (e.g., aciclovir), penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine. In some embodiments, the additional anti-viral therapeutic is selected from the group consisting of lamivudine, an interferon alpha, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), rifampicin, zanamivir, oseltamivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ccoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine. In some embodiments, the another therapeutic is selected from the group consisting of quinine (optionally in combination with clindamycin), chloroquine, amodiaquine, artemisinin and its derivatives (e.g., artemether, artesunate, dihydroartemisinin, arteether), doxycycline, pyrimethamine, mefloquine, halofantrine, hydroxychloroquine, eflornithine, nitazoxanide, ornidazole, paromomycin, pentamidine, primaquine, pyrimethamine, proguanil (optionally in combination with atovaquone), a sulfonamide (e.g., sulfadoxine, sulfamethoxypyridazine), tafenoquine, tinidazole and a PPT1 inhibitor (including Lys05 and DC661). In some embodiments, the another therapeutic is an antibiotic. In some embodiments, the antibiotic is a penicillin antibiotic, a quinolone antibiotic, a tetracycline antibiotic, a macrolide antibiotic, a lincosamide antibiotic, a cephalosporin antibiotic, or an RNA synthetase inhibitor. In some embodiments, the antibiotic is selected from the group consisting of azithromycin, vancomycin, metronidazole, gentamicin, colistin, fidaxomicin, telavancin, oritavancin, dalbavancin, daptomycin, cephalexin, cefuroxime, cefadroxil, cefazolin, cephalothin, cefaclor, cefamandole, cefoxitin, cefprozil, ceftobiprole, cipro, Levaquin, floxin, tequin, avelox, norflox, tetracycline, minocycline, oxytetracycline, doxycycline, amoxicillin, ampicillin, penicillin V, dicloxacillin, carbenicillin, methicillin, ertapenem, doripenem, imipenem/cilastatin, meropenem, amikacin, kanamycin, neomycin, netilmicin, tobramycin, paromomycin, cefixime, cefdinir, cefditoren, cefoperazone, cefotaxime, ceftazidime, ceftibuten, ceftizoxime, ceftriaxone, cefoxotin, and streptomycin. In some embodiments, the antibiotic is azithromycin.

In some embodiments, the one or more additional therapeutic agents that may be administered in combination with a crystalline compound provided herein can be selected from the group consisting of ribavirin, favipiravir, ST-193, oseltamivir, zanamivir, peramivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine.

In some embodiments, the crystalline compounds described herein may be used in combination with one or more other agents which may be useful in the prevention or treatment of respiratory disease, inflammatory disease, autoimmune disease, for example; anti-histamines, corticosteroids, (e.g., fluticasone propionate, fluticasone furoate, beclomethasone dipropionate, budesonide, ciclesonide, mometasone furoate, triamcinolone, flunisolide), NSAIDs, leukotriene modulators (e.g., montelukast, zafirlukast, pranlukast), tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, PI3K delta inhibitors, ITK inhibitors, LP (lysophosphatidic) inhibitors or FLAP (5-lipoxygenase activating protein) inhibitors (e.g., sodium 3-(3-(tert-butylthio)-1-(4-(6-ethoxypyridin-3-yl)benzyl)-5-((5-ethylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-IgE, anti-TNF, anti-IL-5, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents), suitable anti-infective agents including antibiotic agents, antifungal agents, anthelmintic agents, antimalarial agents, antiprotozoal agents and antituberculosis agents.

In some embodiments, the additional therapeutic agents can be kinase inhibitors including but not limited to erlotinib, gefitinib, neratinib, afatinib, osimertinib, lapatanib, crizotinib, brigatinib, ceritinib, alectinib, lorlatinib, everolimus, temsirolimus, abemaciclib, LEE011, palbociclib, cabozantinib, sunitinib, pazopanib, sorafenib, regorafenib, axitinib, dasatinib, imatinib, nilotinib, ponatinib, idelalisib, ibrutinib, Loxo 292, larotrectinib, and quizartinib.

In some embodiments, the additional therapeutic agents can be therapeutic anti-viral vaccines.

In some embodiments, the additional therapeutic agents can be immunomodulatory agents including but not limited to anti-PD-1 or anti-PDL-1 therapeutics including pembrolizumab, nivolumab, atezolizumab, durvalumab, BMS-936559, or avelumab, anti-TIM3 (anti-HA Vcr2) therapeutics including but not limited to TSR-022 or MBG453, anti-LAG3 therapeutics including but not limited to relatlimab, LAG525, or TSR-033, anti-4-1BB (anti-CD37, anti-TNFRSF9), CD40 agonist therapeutics including but not limited to SGN-40, CP-870,893 or RO7009789, anti-CD47 therapeutics including but not limited to Hu5F9-G4, anti-CD20 therapeutics, anti-CD38 therapeutics, STING agonists including but not limited to ADU-S100, MK-1454, ASA404, or amidobenzimidazoles, anthracyclines including but not limited to doxorubicin or mitoxanthrone, hypomethylating agents including but not limited to azacytidine or decitabine, other immunomodulatory therapeutics including but not limited to epidermal growth factor inhibitors, statins, metformin, angiotensin receptor blockers, thalidomide, lenalidomide, pomalidomide, prednisone, or dexamethasone. In some embodiments, the additional therapeutic agent is a p2-adrenoreceptor agonist including, but not limited to, vilanterol, salmeterol, salbutamol. formoterol, salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt of salbutamol or the fumarate salt of formoterol. In some embodiments, the additional therapeutic agent is an anticholinergic agent, including, but not limited to, umeclidinium (for example, as the bromide), ipratropium (for example, as the bromide), oxitropium (for example, as the bromide) and tiotropium (for example, as the bromide).

In particular, in certain embodiments, the disclosure provides a method of treating the above medical indications comprising administering to a subject in need thereof a therapeutically effective amount of a crystalline compound described herein, such as a disclosed crystalline compound.

The term “boosting amount” or “boosting dose” is the amount of a compound needed to improve the pharmacokinetics of a second compound (or increase availability or exposure). The boosting amount or boosting dose may improve the pharmacokinetics (or increase availability or exposure) of the second compound to a level to therapeutic levels in a subject.

In one embodiment, the disclosure provides for a disclosed crystalline compound to be administered together with an antiviral therapeutic such as disclosed herein, and e.g., thereby boosting the dose of the anti-viral therapeutic or therapeutics. Such a boost combination may be used, e.g., as prophylactic or therapeutic treatment of a viral infection in a subject in need thereof. In one embodiment, the protease inhibitor is a crystalline compound described herein.

### EXAMPLES

The compounds described herein can be prepared in a number of ways based on the teachings contained herein and synthetic procedures known in the art. The following non-limiting examples illustrate the disclosure. In some embodiments, a crystalline form described herein converts to another crystalline form upon achieving an onset point or an endothermic peak, measured by differential scanning calorimetry (DSC), which may be higher temperatures than those of the crystalline forms described herein.

X-ray powder diffraction was performed using Bruker D8 Advance and PANalytical AERIS diffractometers according to the parameters listed in Table 1.

TABLE 1
The Parameters of XRPD Diffraction

| Instrument | PANalytical AERIS | Bruker, D8 Advance | |
|---|---|---|---|
| Radiation | Cu Kα | Cu Kα | |
| | (λ = 1.5418 Å) | (λ = 1.5418 Å) | |
| Detector | PIXcellD-Medipix3 | LynxEye | |
| Scan angle | 3-40° (2θ) | 3-40° (2θ) | |
| Scan step | 0.02° (2θ) | 0.02° (2θ) | |
| Scan speed | 0.11 s/step | 0.2 | s/step |
| Tube | 40 KV/7.5 mA | 40 KV/40 mA | |
| voltage/current | | | |
| Divergence slit | 1/16° | 0.6 | mm |

| | | |
|---|---|---|
| Rotation | On | On |
| Sample | Zero-background | Zero-background |
| holder | sample pan | sample holder |

Differential scanning calorimetry (DSC) was performed according to the parameters listed in Table 2.

TABLE 2
The Parameters of DSC Analysis

| Instrument | TA, Q200/Discovery DSC | |
|---|---|---|
| | 250 | |
| Sample pan | Aluminum, pin-holed | |
| Temperature | 25-250/300° C. | |
| range | | |
| Heating rate | 10° | C./min |
| Purge gas | N2 | |
| Flow rate | 50 | mL/min |

Thermal gravimetric analysis (TGA) was performed according to the parameters listed in Table 3.

TABLE 3
The Parameters of TGA Analysis

| | |
|---|---|
| Instrument | TA, Q500/Discovery TGA 55 |
| Sample pan | Aluminum, open |
| Temperature | RT-250/300° C. |
| range | |
| Heating rate | 10° C./min |
| Purge gas | N2 |
| Flow rate | Balance chamber: 40 mL/min |
| | Sample chamber: 60 mL/min |

Dynamic vapor sorption (DVS) analysis was performed according to the parameters listed in Table 4.

TABLE 4
The Parameters of DVS Analysis

| Instrument | SMS, DVS Intrinsic | |
|---|---|---|
| Step time | 60 | min |
| Drying/Measurement | 40° C./25° C. | |
| temperature | | |
| Cycle | Full cycle | |
| Step time for each RH % | 1 | hour |
| Save data rate | 5 | s |
| Total flow rate | 200 | sccm |
| Post experiment total | 200 | sccm |

| | |
|---|---|
| flow | |
| Method 1 | Adsorption: 0, 10, 20, 30, 40, 50, 60, 70, 80, 90 |
| | Desorption: 80, 70, 60, 50, 40, 30, 20, 10, 0 |
| Method 2 | Adsorption 1: 30, 40, 50, 60, 70, 80, 90 |
| | Desorption 1: 80, 70, 60, 50, 40, 30, 20, 10, 0 |
| | Adsorption 2: 0, 10, 20, 30, 40, 50, 60, 70, 80, 90 |
| | Desorption 2: 80, 70, 60, 50, 40, 30, 20, 10, 0 |

### Example 1

Crystalline Form C (7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate) was prepared by adding a mixed solvent of isopropanol/water (0.7 mL/0.7 mL) to a vial with 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide (80 mg) to achieve a gel. The gel was then stirred at 50° C. for 3 days. The resulting solids were filtered and dried under vacuum at 50° C. overnight.

Alternatively, crystalline Form C (7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate) was prepared by adding a mixed solvent of methanol/n-propyl acetate in 1:1 or 1:2 ratio to a vial with 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide to achieve a gel. The gel was then stirred. The resulting solids were filtered and dried under vacuum.

Alternatively, crystalline Form C (7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate) was prepared by adding a mixture of acetic acid and acetone to a vial with 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide to achieve a slurry. The mixture was added with more acetone at about 20-25° C. to form a solid. Then, the slurry was heated to about 53-58° C. and stirred for about 24-25 hours to form a suspension. The suspension was cooled to about 20-25° C. over 1.5-2 hours, and then stirred for another about 3 hours. The solids were filtered, washed with acetone, and dried to form solids under vacuum at about 50-55° C. for about 15 hours.

Alternatively, crystalline Form C (7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate) was prepared by adding a mixture of acetic acid and acetone to a vial with 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide to achieve a slurry. The mixture was added with more acetone at about 20-25° C. to form a solid. Then, the slurry was heated to about 53-58° C. and stirred for about 24-25 hours to form a suspension. The suspension was cooled to about 20-25° C. over 1.5-2 hours, and then stirred for another about 3 hours. The solids were filtered, washed with acetone, and dried to form solids under vacuum at about 50-55° C. for about 15 hours. And then, water was added to a vial with 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide to achieve a slurry. The slurry was then stirred at about 20-25° C. Seeds of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide in water were submitted, and then the solids were stirred at about 68-73 ºC for about 24-25 hours. The solids were then cooled about 20-25° C. for about 2.5-3 hours, and then stirred for about 22 hours. The solids were filtered, washed with water, and then dried under vacuum at about 58-63° C. for about 24 hours.

Alternatively, crystalline Form C (7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate) was prepared by adding water to a vial with 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide to achieve a slurry. The slurry was then stirred for about 12 hours at about 60° C. (55-65° C.). The slurry was then cooled to about 20° C. (15-25° C.) for about 4 hours (2 to 6 hours), and then the resulting solids were washed, filtered, and dried under vacuum at about 45° C. (40-50° C.) for about 16 hours (14-18 hours).

Alternatively, crystalline Form C (7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate) was prepared by adding water to a vial with 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide to achieve a slurry. The slurry was then stirred for about 12 hours at about 60° C. (55-65° C.). The slurry was then cooled to about 20° C. (15-25° C.) for about 4 hours (2 to 6 hours), and then the resulting solids were washed, filtered, and dried under vacuum at about 45° C. (40-50° C.) for about 16 hours (14-18 hours). And then, the resulting solid was dried for another about 8 hours (6-10 hours) at about 45° C. (40-50° C.).

Crystalline Form F (7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate) was prepared by adding methylethylketone (1.6 mL) to a vial of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide (100 mg) to achieve a suspension. The suspension was then stirred at room temperature for 3 days. The resulting solids were filtered and dried under ambient condition overnight.

Alternatively, crystalline Form F (7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate) was prepared by adding water (1% of total solution), methylethylketone (0.4 mL) to a vial of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide (20 mg) to achieve a mixture and then slurrying the mixture at room temperature for 3 days. The resulting solids were filtered and dried under ambient condition overnight.

### Example 2

The XRPD pattern of crystalline Form A is shown in FIG. 1. Characteristic peaks include one or more of the peaks shown in Table 5.

TABLE 5
XRPD pattern of crystalline Form A

| Angle | Intensity % | Intensity | d value |
|---|---|---|---|
| (2-Theta °) | (%) | (Count) | (Angstrom) |
| 6.6 | 37.9 | 1838 | 13.34 |
| 9.8 | 44.2 | 2143 | 8.97 |
| 13.9 | 32.5 | 1574 | 6.36 |
| 14.6 | 52.4 | 2540 | 6.07 |
| 15.1 | 44 | 2135 | 5.86 |
| 16.8 | 30.8 | 1495 | 5.26 |
| 18.5 | 28.1 | 1362 | 4.78 |
| 19.7 | 55.3 | 2681 | 4.50 |
| 21.1 | 35.6 | 1726 | 4.21 |
| 24.3 | 100 | 4847 | 3.66 |

FIG. 2 depicts the differential scanning calorimetry (DSC) profile of crystalline Form A. As shown in FIG. 2, crystalline Form A shows a characteristic endotherm with an onset of about 102° C. and a peak of about 116° C.

Crystalline Form A, 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methanol solvate, displayed a thermogravimetric analysis (TGA) profile showing a mass loss of about 5.5 wt. % up to about 150° C.

### Example 3

The XRPD pattern of crystalline Form B is shown in FIG. 3. Characteristic peaks include one or more of the peaks shown in Table 6.

TABLE 6
XRPD pattern of crystalline Form B

| Angle | Intensity % | Intensity | d value |
|---|---|---|---|
| (2-Theta °) | (%) | (Count) | (Angstrom) |
| 6.5 | 45.7 | 1729 | 13.64 |
| 7.1 | 41.2 | 1560 | 12.43 |
| 7.9 | 58 | 2193 | 11.14 |
| 8.4 | 44.5 | 1682 | 10.56 |
| 13.9 | 88.4 | 3346 | 6.38 |
| 14.7 | 43.5 | 1646 | 6.02 |
| 17.5 | 57.3 | 2167 | 5.05 |
| 20.3 | 65.7 | 2484 | 4.38 |
| 22.9 | 48.9 | 1849 | 3.89 |
| 24.2 | 100 | 3783 | 3.67 |

FIG. 4 depicts the differential scanning calorimetry (DSC) profile of crystalline Form B. As shown in FIG. 4, crystalline Form B shows a characteristic endotherm with an onset of about 52° C., and a peak of about 118° C.

Crystalline Form B, 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate, displayed a thermogravimetric analysis (TGA) profile showing a mass loss of about 8.1 wt. % up to about 150° C.

### Example 4

The XRPD pattern of crystalline Form C is shown in FIG. 5. Characteristic peaks include one or more of the peaks shown in Table 7.

TABLE 7
XRPD pattern of crystalline Form C

| Angle | Intensity % | Intensity | d value |
|---|---|---|---|
| (2-Theta °) | (%) | (Count) | (Angstrom) |
| 3.3 | 62.6 | 1882 | 26.44 |
| 3.7 | 43.3 | 1303 | 23.68 |
| 4.8 | 40.1 | 1207 | 18.44 |
| 5.4 | 63.3 | 1904 | 16.48 |
| 6.1 | 39.4 | 1184 | 14.43 |
| 7.0 | 100 | 3007 | 12.60 |
| 7.2 | 59.1 | 1778 | 12.21 |
| 18.5 | 26.2 | 787 | 4.80 |
| 22.9 | 18.3 | 550 | 3.89 |
| 24.2 | 18.6 | 559 | 3.68 |

FIG. 6 depicts the differential scanning calorimetry (DSC) profile of crystalline Form C. As shown in FIG. 6, crystalline Form C shows a characteristic endotherm with an onset of about 171 to about 193° C., and a peak of about 180 to about 197° C.

Crystalline Form C, 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate, displayed a thermogravimetric analysis (TGA) profile showing a mass loss of about 0.8 wt. % up to about 165° C. Crystalline Form C displayed a dynamic vapor sorption (DVS) profile showing about 0.5% gain to about 90% relative humidity (RH) at 25° C., and a reversible total mass change of about 0.7% from about 90% to about 0% RH.

Approximate solubility was determined by visual observation and shown in Form C Table 8.

TABLE 8
Solubility of Form C

| Solvent | Solubility of Form C (mg/mL) |
|---|---|
| Methanol | >9 |
| Ethanol | >2 |
| Acetone | <2 |
| Methanol/Dichloromethane (1/1) | >23 |
| Methanol/Dichloromethane (1/2) | >28 |
| Methanol/Dichloromethane (2/1) | >16 |

### Example 5

The XRPD pattern of crystalline Form D is shown in FIG. 7. Characteristic peaks include one or more of the peaks shown in Table 9.

TABLE 9
XRPD pattern of crystalline Form D

| Angle | Intensity % | Intensity | d value |
|---|---|---|---|
| (2-Theta °) | (%) | (Count) | (Angstrom) |
| 6.3 | 100 | 11823 | 14.02 |
| 7.0 | 7.3 | 864 | 12.69 |
| 11.5 | 7.9 | 932 | 7.66 |
| 12.6 | 6.2 | 730 | 7.02 |
| 14.2 | 7.2 | 847 | 6.21 |
| 17.3 | 19 | 2242 | 5.12 |
| 18.2 | 11.1 | 1313 | 4.87 |
| 18.8 | 7.5 | 892 | 4.73 |
| 19.2 | 7.1 | 844 | 4.62 |
| 25.2 | 9.3 | 1094 | 3.53 |

FIG. 8 depicts the differential scanning calorimetry (DSC) profile of crystalline Form D. As shown in FIG. 8, crystalline Form D shows a characteristic endotherm with an onset of about 115° C. and a peak of about 125° C.

Crystalline Form D, 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl ethyl ketone solvate, displayed a thermogravimetric analysis (TGA) profile showing a mass loss of about 15.5 wt. % up to about 150° C.

### Example 6

The XRPD pattern of crystalline Form E is shown in FIG. 9. Characteristic peaks include one or more of the peaks shown in Table 10.

TABLE 10
XRPD pattern of crystalline Form E

| Angle | Intensity % | Intensity | d value |
|---|---|---|---|
| (2-Theta °) | (%) | (Count) | (Angstrom) |
| 6.2 | 100 | 49909 | 14.23 |
| 12.5 | 1.8 | 896 | 7.06 |
| 12.9 | 12.4 | 6188 | 6.87 |
| 13.7 | 1.8 | 896 | 6.46 |
| 14.7 | 7.7 | 3865 | 6.02 |
| 14.9 | 5.7 | 2843 | 5.93 |
| 17.0 | 1.7 | 824 | 5.20 |
| 19.0 | 1.7 | 857 | 4.68 |
| 19.8 | 7.2 | 3597 | 4.49 |
| 38.4 | 1.8 | 893 | 2.34 |

FIG. 10 depicts the differential scanning calorimetry (DSC) profile of crystalline Form E. As shown in FIG. 10, crystalline Form E shows a characteristic endotherm with an onset of about 105° C. and a peak of about 113° C.; and a characteristic endotherm with an onset of about 174° C. and a peak of about 186° C.

Crystalline Form E 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl isobutyl ketone solvate, displayed a thermogravimetric analysis (TGA) profile showing a mass loss of about 11.8 wt. % up to about 125° C.

### Example 7

The XRPD pattern of crystalline Form F is shown in FIG. 11. Characteristic peaks include one or more of the peaks shown in Table 11.

TABLE 11
XRPD pattern of crystalline Form F

| Angle | Intensity % | Intensity | d value |
|---|---|---|---|
| (2-Theta °) | (%) | (Count) | (Angstrom) |
| 6.9 | 40.2 | 999 | 12.72 |
| 7.8 | 94 | 2333 | 11.33 |
| 8.2 | 61.9 | 1537 | 10.72 |
| 13.7 | 82.2 | 2041 | 6.44 |
| 14.6 | 78.3 | 1944 | 6.08 |
| 17.4 | 51.4 | 1275 | 5.09 |
| 18.5 | 37.6 | 932 | 4.80 |
| 20.1 | 93 | 2308 | 4.41 |
| 22.7 | 54.3 | 1347 | 3.92 |
| 24.1 | 100 | 2482 | 3.69 |

FIG. 12 depicts the differential scanning calorimetry (DSC) profile of crystalline Form F. As shown in FIG. 12, crystalline Form F shows a characteristic endotherm with an onset of about 51 to about 65° C., and a peak of about 79 to about 91° C.; and a characteristic endotherm with an onset of about 117 to about 119° C., and a peak of about 127 to about 128° C.

Crystalline Form F 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate, displayed a thermogravimetric analysis (TGA) profile showing a mass loss of 2.7 wt. % up to about 100° C. Crystalline Form F displayed a dynamic vapor sorption (DVS) profile showing a reversable gain of about 4 wt. % with no hysteresis from about 0 to about 90% relative humidity (RH).

Approximate solubility was determined by visual observation and shown in Form F Table 12.

TABLE 12
Solubility of Form F

| Solvent | Solubility of Form F (mg/mL) |
|---|---|
| Methanol | >11 |
| Ethanol | >7 |
| Acetone | <2 |
| Methanol/Dichloromethane (1/1) | >47 |
| Methanol/Dichloromethane (1/2) | >58 |
| Methanol/Dichloromethane (2/1) | >19 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, anhydrate (Form C), characterized by a powder X-ray diffraction pattern having a characteristic peak in degrees 2θ at about 5.4.

2. The crystalline compound of claim 1, having an XRPD pattern with characteristic peaks at the following values of 20 in degrees at about 3.3, 3.7, and 5.4.

3. The crystalline compound of claim 1 or 2, having an XRPD pattern with characteristic peaks at the following values of 20 in degrees at about 3.3, 3.7, 5.4, 7.0 and 7.2.

4. The crystalline form of any one of claims 1-3, wherein the powder X-ray diffraction pattern was obtained using Cu Kα radiation.

5. The crystalline form of any one of claims 1-4, characterized by a differential scanning calorimetry (DSC) profile having a characteristic endotherm with an onset of about 171 to about 193° C., and a peak of about 180 to about 197° C.

6. A crystalline monohydrate form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate (Form F), characterized by a powder X-ray diffraction pattern having a characteristic peak in degrees 2θ at about 8.2.

7. The crystalline compound of claim 6, having an XRPD pattern with characteristic peaks at the following values of 20 in degrees at about 7.8, 8.2, and 13.7.

8. The crystalline compound of claim 6 or 7, having an XRPD pattern with characteristic peaks at the following values of 20 in degrees at about 7.8, 8.2, 13.7, 14.6, 20.1, and 24.1.

9. The crystalline form of any one of claims 6-8, wherein the powder X-ray diffraction pattern was obtained using Cu Kα radiation.

10. The crystalline form of any one of claims 6-9, characterized by a differential scanning calorimetry (DSC) profile having a characteristic endotherm with an onset of about 51 to about 65° C., and a peak of about 79 to about 91° C.

11. The crystalline form of any one of claims 6-10, characterized by a differential scanning calorimetry (DSC) profile having a characteristic endotherm with an onset of about 117 to about 119 ºC, and a peak of about 127 to about 128° C.

12. A crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methanol solvate (Form A), characterized by a powder X-ray diffraction pattern having a characteristic peak in degrees 2θ at about 9.8, wherein the powder X-ray diffraction pattern was obtained using Cu Kα radiation.

13. The crystalline compound of claim 12, having an XRPD pattern with characteristic peaks at the following values of 20 in degrees at about 9.8, 14.6, 15.1, 19.7, and 24.3.

14. The crystalline form of claim 12 or 13, characterized by a differential scanning calorimetry (DSC) profile having a characteristic endotherm with an onset of about 102° C. and a peak of about 116° C.

15. A crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, hydrate (Form B), characterized by a powder X-ray diffraction pattern having a characteristic peak in degrees 2θ at about 6.5 and 8.4, wherein the powder X-ray diffraction pattern was obtained using Cu Kα radiation.

16. The crystalline compound of claim 15, having an XRPD pattern with characteristic peaks at the following values of 20 in degrees at about 6.5, 7.9, 8.4, 13.9, 17.5, 20.3, and 24.2.

17. The crystalline form of claim 15 or 16, characterized by a differential scanning calorimetry (DSC) profile having a characteristic endotherm with an onset of about 52° C. and a peak of about 118° C.

18. A crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl ethyl ketone solvate (Form D), characterized by a powder X-ray diffraction pattern having a characteristic peak in degrees 2θ at about 6.3 and 11.5, wherein the powder X-ray diffraction pattern was obtained using Cu Kα radiation.

19. The crystalline compound of claim 18, having an XRPD pattern with characteristic peaks at the following values of 20 in degrees at about 6.3, 11.5, 17.3, 18.2, and 25.2.

20. The crystalline form of claim 18 or 19, characterized by a differential scanning calorimetry (DSC) profile having a characteristic endotherm with an onset of about 115° C. and a peak of about 125° C.

21. A crystalline form of 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide, methyl isobutyl ketone solvate (Form E), characterized by a powder X-ray diffraction pattern having a characteristic peak in degrees 2θ at about 12.9, wherein the powder X-ray diffraction pattern was obtained using Cu Kα radiation.

22. The crystalline compound of claim 21, having an XRPD pattern with characteristic peaks at the following values of 20 in degrees at about 6.2, 12.9, 14.7, 14.9, and 19.8.

23. The crystalline form of claim 21 or 22, characterized by a differential scanning calorimetry (DSC) profile having a characteristic endotherm with an onset of about 105° C. and a peak of about 113° C.; and a characteristic endotherm with an onset of about 174° C. and a peak of about 186° C.

24. A pharmaceutical composition comprising a crystalline compound of any one claims 1-23, and a pharmaceutically acceptable excipient.

25. A method for making the crystalline form of claim 1 (Form C), the method comprising
contacting an amorphous form of a compound of Formula I with a solvent to form a solubilized form or gel;
stirring the solubilized form or gel; and
drying the solubilized form or gel to form the crystalline form;
wherein the compound of Formula I is 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide.

26. The method of claim 25, wherein the solvent comprises isopropanol.

27. The method of claim 25 or 26, wherein the solvent comprises isopropanol and water.

28. The method of claim 25, wherein the solvent is water.

29. The method of any one of claims 25-28, wherein the stirring is performed at a temperature of about 25-80° C.

30. The method of any one of claims 25-28, wherein drying comprises drying under reduced pressure.

31. A method for making the crystalline form of claim 1 (Form C), the method comprising
contacting an amorphous form, a solvate form, a hydrate form, or a mixture thereof of a compound of Formula I with a solvent to form a solubilized form, a gel or a solid;
stirring the solubilized form, the gel, or the solid; and
drying the solubilized form, the gel, or the solid to form the crystalline form;
wherein the compound of Formula I is 7-chloro-N—((S)-1-(((S)-1-cyano-2-((S)-2-oxopiperidin-3-yl)ethyl)amino)-3-cyclopropyl-1-oxopropan-2-yl)-1H-indole-2-carboxamide.

32. The method of claim 31, wherein the solvate form is an acetone solvate.

33. The method of claim 31, wherein the solvent comprises isopropanol.

34. The method of claim 31, wherein the solvent comprises isopropanol and water.

35. The method of claim 31, wherein the solvent is water.

36. The method of any one of claims 31-35, wherein the stirring is performed at a temperature of about 25-80° C.

37. The method of any one of claims 31-36, wherein drying comprises drying under reduced pressure.

38. A method of ameliorating or treating a viral infection in a patient in need thereof, comprising administering to the patient a therapeutically effective amount of a crystalline form of any one of claims 1-23, or a pharmaceutically acceptable composition of claim 24.

39. The method of claim 38, wherein the viral infection is from a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, arenavirus, a herpes virus, and a hepatovirus.

40. The method of claim 38, wherein the viral infection is from a virus selected from the group consisting of Norwalk virus, feline calicivirus, MD145, murine norovirus, vesicular exanthema of swine virus, rabbit hemorrhagic disease virus, enterovirus (EV)-68 virus, EV-71 virus, poliovirus, coxsackievirus, foot-and-mouth disease virus, hepatitis A, porcine teschovirus, rhinovirus, human coronavirus, transmissible gastroenteritis virus, murine hepatitis virus, bovine coronavirus, feline infectious peritonitis virus, and severe acute respiratory syndrome coronavirus.

41. The method of any one of claims 38-40, wherein the viral infection is a coronavirus infection.

42. The method of any one of claims 38-41, wherein the viral infection is a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19).

43. The method of any one of claims 38-42, wherein the viral infection is SARS-CoV-2.

44. The method of any one of claims 38-43, wherein the viral infection is an arenavirus infection.

45. The method of claim 43, wherein the arenavirus is selected from the group consisting of: Junin virus, Lassa virus, Lujo virus, Machupo virus, and Sabia virus.

46. The method of claim 39, wherein the viral infection is an influenza infection.

47. The method of claim 46, wherein the influenza is influenza H1N1, H3N2 or H5N1.

48. A method of inhibiting transmission of a virus, a method of inhibiting viral replication, a method of minimizing expression of viral proteins, or a method of inhibiting virus release, comprising administering a therapeutically effective amount of a crystalline form of any one of claims 1-23 or a pharmaceutically acceptable composition of claim 24 to a patient suffering from the virus, and/or contacting an effective amount of a crystalline form of any one of claims 1-23 or a pharmaceutically acceptable composition of claim 24.

49. The method of any one of claims 38-48, further comprising administering another therapeutic.

50. The method of any one of claims 38-48, further comprising administering an additional anti-viral therapeutic.

51. The method of claim 50, wherein the anti-viral therapeutic is selected from the group consisting of molnupiravir, remdesivir, ribavirin, favipiravir, ST-193, oseltamivir, zanamivir, peramivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine.

52. The method of claim 49, wherein the another therapeutic is selected from the group consisting of protease inhibitors, fusion inhibitors, M2 proton channel blockers, polymerase inhibitors, 6-endonuclease inhibitors, neuraminidase inhibitors, reverse transcriptase inhibitor, aciclovir, acyclovir, protease inhibitors, arbidol, atazanavir, atripla, boceprevir, cidofovir, combivir, darunavir, docosanol, edoxudine, entry inhibitors, entecavir, famciclovir, fomivirsen, fosamprenavir, foscarnet, fosfonet, ganciclovir, ibacitabine, immunovir, idoxuridine, imiquimod, inosine, integrase inhibitor, interferons, lopinavir, loviride, moroxydine, nexavir, nucleoside analogues, penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine.

53. The method of claim 50, wherein the additional anti-viral therapeutic is selected from the group consisting of lamivudine, an interferon alpha, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), rifampicin, zanamivir, oseltamivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine.

54. A method of prophylactically treating a patient at risk of viral infection, comprising administering to the patient an effective amount of a crystalline form of any one of claims 1-23, or a pharmaceutically acceptable composition of claim 24.

55. The method of claim 54, wherein the compound is administered before viral exposure.

56. The method of claim 54, wherein the compound is administered after viral exposure.
